(19) United States
(12) Patent Application Publication
(10) Pub. No.: US 2021/0298583 A1
(43) Pub. Date: Sep. 30, 2021

(54) ENDOSCOPE WITH SECONDARY WORKING CHANNEL

(71) Applicants: Hasnain Bawaadam, Vernon Hills, IL (US); Fazila Barodawala, Vernon Hills, IL (US); R. Daniel Maynes, Provo, UT (US)

(72) Inventors: Hasnain Bawaadam, Vernon Hills, IL (US); Fazila Barodawala, Vernon Hills, IL (US); R. Daniel Maynes, Provo, UT (US)

(21) Appl. No.: 17/346,247

(22) Filed: Jun. 13, 2021

Related U.S. Application Data
(63) Continuation-in-part of application No. 15/931,586, filed on May 14, 2020, now abandoned.
Application No. 15/931,586 is a continuation-in-part of application No. 16/274,222, filed on Feb. 12, 2019, now abandoned.
Application No. 16/274,222 is a continuation-in-part of application No. PCT/US2017/046665, filed on Aug. 12, 2017.
(60) Provisional application No. 62/374,015, filed on Aug. 12, 2016.

(51) Int. Cl. A61B 1/015; A61B 1/018; A61B 1/005; A61B 1/267

(52) U.S. Cl. CPC A61B 1/015; A61B 1/2676; A61B 1/005; A61B 1/018

(57) ABSTRACT

An endoscope including an examination field cleaning system and a method of using the endoscope to clean an examination field in an internal body cavity are disclosed herein. The endoscope includes an inner channel that includes a primary working channel, lens system, and light delivery system and an outer secondary working channel that includes an examination field cleaning system. The inner channel is approximately cylindrical and is encompassed by a flexible tube. The outer secondary working channel is encompassed by an outer cylinder that is approximately concentric with and fully encompasses the inner channel and tube, where the examination field cleaning system may include one or more fluid delivery channels. The endoscope may further have an anchoring bridge that secures the outer cylinder to the inner channel.

### CROSS-REFERENCE TO RELATED APPLICATIONS

This application is a continuation-in-part of U.S. patent application Ser. No. 15/931,586, filed on May 14, 2020, which is a continuation-in-part of U.S. patent application Ser. No. 16/274,222, filed on Feb. 12, 2019, which is a continuation-in-part of PCT Patent Application No. PCT/US2017/046665, filed on Aug. 12, 2017, which claims the benefit of and priority to U.S. Provisional Patent Application Ser. No. 62/374,015, filed on Aug. 12, 2016, the disclosures of which are incorporated herein in their entireties by reference.

### BACKGROUND

### Field of the Invention

This disclosure relates to an endoscope with a secondary channel allowing for the continuous instillation of water or an aqueous cleaning solution.

### Description of the Related Art

An endoscope is a widely used minimally invasive medical device that permits a user to investigate symptoms, confirm a diagnosis, or provide treatment to a patient. The user is typically a physician or other qualified health care provider. Endoscopes are used in a wide variety of examination and treatment procedures, including examination or treatment of the gastrointestinal tract using a device such as an esophagogastroduodenoscope, enteroscope, or colonoscope; examination or treatment of the respiratory tract using a device such as a rhinoscope, bronchoscope, or otoscope; examination or treatment of the urinary tract using a cystoscope; examination or treatment of the female reproductive system using a device such as a colposcope, hysteroscope, or falloposcope; and examination or treatment of normally closed body cavities using a device such as a laparoscope, arthroscope, thoracoscope, or mediastinoscope. Each type of endoscope is designed and manufactured to accommodate the specific needs and limitations of the particular area of the body in which it will be used.

An endoscope typically includes a rigid or flexible tube; a light delivery system for illumination of the organ, tissue, or other object to be examined or treated, where the light source may be outside the body and where the light may be delivered to the examination field by an optical fiber system; a camera system or other lens system that transmits an image of the examination field to the user or other viewer; and a working channel for delivery of medical instruments.

Certain procedures using an endoscope will require excision of a small tissue sample for biopsy. A common problem encountered in such procedures is obscuring of the examination field. This is typically caused by bleeding or release of other fluids from the excision site, where the blood or other fluid released prevents the user from clearly observing the examination field, and thus preventing additional biopsies if necessary. This problem may be alleviated by the delivery of water or an aqueous cleaning solution to the examination field, clearing debris and improving clarity of the examination field. However, the water or an aqueous cleaning solution must actually wash away the blood or other fluid obscuring the examination field without further obscuring the examination field, and must also not damage the tissue being examined.

U.S. Patent Application Publication No. 2007/0260113 discloses an endoscope including a secondary channel for introducing air, water, or a cleaning liquid to the examination field. U.S. Pat. No. 8,333,690 discloses a fluid feed system for an endoscope including a component for injection of a fluid to clean the examination field. These and other disclosed endoscopic examination field cleaning systems employ a single tube with a circular opening for delivery of a fluid. U.S. Pat. No. 8,888,683 discloses an intubating scope designed for therapeutic use that has multiple secondary channels with approximately circular or semicircular openings that are situated on the exterior of the primary working channel and that may be used for delivery of a cleaning fluid. These cleaning systems suffer from various limitations. For example, in endoscopes with circular secondary channels for fluid instillation, at low fluid flow rates the surface tension of the fluid causes the fluid to exit the secondary channel as droplets rather than as a spray, thus often leading to further obscuring of the examination field rather than the desired cleaning effect. If the fluid flow rate is increased to exceed the threshold at which droplets will form, the impact pressure of fluid on the tissue being examined may damage the tissue.

Thus there remains a need for an endoscope with an examination field cleaning system that allows for introduction of a cleaning fluid in a way that does not cause further obscuring of the examination field or damage to the tissue being examined.

### SUMMARY

An endoscope that includes an examination field cleaning system is disclosed herein. The endoscope includes an inner channel that includes a primary working channel, lens system, and light delivery system and an outer secondary working channel that includes an examination field cleaning system. The inner channel has an approximately cylindrical shape and is encompassed by a flexible tube. The outer secondary working channel is configured to be encompassed by an outer cylinder that is approximately concentric with and fully encompasses the inner channel and tube, where the examination field cleaning system may include one or more fluid delivery channels. The endoscope may further include an anchoring bridge that secures the outer cylinder to the inner channel. The anchoring bridge may extend the length of the endoscope or may be composed of a multi-part system including a primary anchoring bridge segment extending most of the length of the endoscope except near the distal end and a secondary anchoring bridge segment at the distal end of the endoscope.

A method of using the disclosed endoscope to clean an examination field in an internal body cavity is also described herein. A cleaning fluid may be introduced, via one or more fluid delivery channels that are configured to form an annular secondary channel, into the examination field to wash away blood or other fluids that are obscuring the examination field.

### BRIEF DESCRIPTION OF THE DRAWINGS

FIG. 1 shows a distal view of an embodiment of the endoscope.

FIG. 2 shows a comparison of distal views of an embodiment of the disclosed endoscope shown in FIG. 2A and a prior art endoscope shown in FIG. 2B.

FIG. 3 shows a distal view of an embodiment of the endoscope with an anchoring bridge.

FIG. 4 shows an embodiment of the endoscope where the anchoring bridge extends the length of the endoscope.

FIG. 5 shows an embodiment of the endoscope where the anchoring bridge includes a primary anchoring bridge segment and a secondary anchoring bridge segment.

FIG. 6 shows an embodiment of the endoscope with an anchoring bridge that extends the entire length of the main body segment.

FIG. 7 shows an embodiment of the endoscope with a primary anchoring bridge segment that extends the length of the main body segment, a secondary anchoring bridge segment that extends the length of the distal end segment, and a position adjustment segment separating the primary and secondary anchoring bridge segments.

### DETAILED DESCRIPTION OF THE ILLUSTRATED EMBODIMENTS

An endoscope that includes an examination field cleaning system is disclosed herein. The endoscope includes an inner channel that includes a primary working channel, lens system, and light delivery system and an outer secondary channel that includes an examination field cleaning system. The inner channel has an approximately cylindrical shape and is encompassed by a flexible tube. The outer secondary channel is configured to be encompassed by an outer cylinder that is approximately concentric with and fully encompasses the inner channel and tube, where the examination field cleaning system may include one or more fluid delivery channels.

The primary working channel of the endoscope is configured to allow insertion of a medical instrument there-through. The medical instrument may, for example, be a forceps, brush, probe, needle, balloon, or the like. The instrument may be used for diagnosis, therapy, or both. Thus, the endoscope is configured for both diagnostic and therapeutic uses.

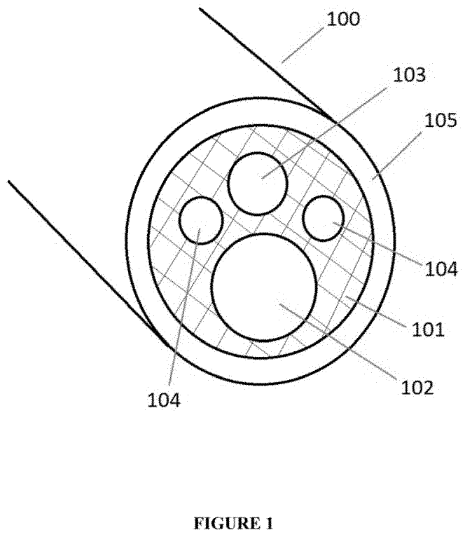

FIG. 1 shows a distal view of an embodiment 100 of the disclosed endoscope, including an inner channel 101 that has a primary working channel 102, a lens system 103, and light sources 104, and also including an outer secondary channel 105 that consists of a single annular fluid delivery channel. Water or another cleaning fluid such as an aqueous saline solution is instilled via outer secondary channel 105. The flow pattern allows the user to introduce the cleaning fluid at a desired flow rate while significantly reducing the likelihood that the cleaning fluid will form droplets that will further obscure the examination field rather than cleaning it as desired. The outer secondary channel 105 may be attached to a cleaning fluid storage vessel, where a valve controls the rate at which cleaning fluid enters the outer secondary channel from the cleaning fluid storage vessel. The flow rate of the cleaning fluid may optionally be controlled by a foot pedal, where the foot pedal is attached to the cleaning fluid storage vessel and where activation of the foot pedal actuates the valve.

In some embodiments, the endoscope is a bronchoscope, rhinoscope, enteroscope, esophagogastroduodenoscope, or a cystoscope. In applications where the overall diameter of the endoscope is limited by its use, such as for a bronchoscope, cystoscope, or other endoscope used in a narrow passage, the outer secondary channel eliminates the need for an additional tube within the inner channel that will not only significantly increase the overall diameter of the inner channel and thereby significantly increase the overall diameter of the endoscope but also compromise the diameter of the primary working channel. By distributing the flow of cleaning fluid substantially uniformly around the inner channel, the disclosed endoscope has a decreased overall diameter as compared to previously disclosed endoscopes that have an examination field cleaning system.

With respect to bronchoscopy, for example, it is well understood by pulmonologists that different types of bronchoscopes are suited for different applications. The respiratory tract has multiple zones that are defined by successive divisions or generations of airway branching. In one characterization, the respiratory tract may be divided into a conducting zone, which represents the 1^{st} through 16^{th} divisions of the respiratory tract, and transitional and respiratory zones, which represent the 17^{th} through 23^{rd} divisions of the respiratory tract. Small airways are defined as having a diameter of less than 2 mm and arise between about the 4^{th} and 13^{th} generations of airway branching. On average, the small airways arise at about the 8^{th} generation of airway branching. See Weibel, E. R. Morphometry of the Human Lung. New York: Academic Press Inc., 1963. 110-35. Segmental bronchii, which are the 3^{rd} generation of airway branching, typically have a diameter ranging between about 4.5 and 13 mm. Subsegmental bronchii, which are the 4^{th} through 9^{th} generations of airway branching, typically have a diameter ranging between about 1 and 6 mm. See Kacmarek, R. M., el al. Essentials of Respiratory Care. Elsevier Health Sciences, 2013, ISBN 978-032327778-5.

A rigid bronchoscope may be used to enter the first generation of airway branching. To enter significantly further into the respiratory tract requires the use of a flexible bronchoscope which has a narrower overall diameter and the flexibility required to enter the branched airways of the respiratory tract. Complete dexterity is required for maneuverability during diagnostic and therapeutic bronchoscopy. In bronchoscopy, such dexterity is only provided by flexible bronchoscopes. This also prevents complications such as airway trauma and bleeding.

Flexible bronchoscopes are available from several manufacturers in a variety of sizes. The typical pediatric flexible bronchoscopes currently in use have an outer diameter of 2.8 mm and a suction channel with a diameter of about 1.2 mm. Smaller instruments, such as instruments having an outer diameter of 2.2 mm, are also available but have no suction channel, and therefore have somewhat limited utility when there are secretions or blood in the airways. These smaller diameter bronchoscopes also cannot be used to obtain diagnostic specimens. Larger bronchoscopes, having an outer diameter ranging between 4.2 mm and 6.3 mm, are used in adults. These bronchoscopes have suction channels that have a diameter ranging between about 2.0 and 3.2 mm. See Wood, R. E. “Bronchoscopy and Bronchoalveolar Lavage in Pediatric Patients,” in Kendig's Disorders of the Respiratory Tract in Children. 9^{th} Ed., 2019.

It is well understood in endoscopy that flexible endoscopes and rigid or semi-rigid endoscopes differ significantly. A flexible endoscope is used to enter passages that require significant dexterity such that the endoscope does not damage the passage while being used therein. By contrast, a rigid or semi-rigid endoscope is used for different applications where the rigidity or semi-rigidity is advantageous, such as applications which require the introduction of multiple instruments at the same time. As described below, there are additional design parameters that must be considered to securely affix an annular outer secondary channel to the inner working channel of a flexible endoscope in a way that maintains the flexibility of the endoscope such that it may be used in applications that require substantial dexterity of the endoscope as compared to the design of an annular outer secondary channel for a rigid endoscope.

In some preferred embodiments, the endoscope is a flexible bronchoscope.

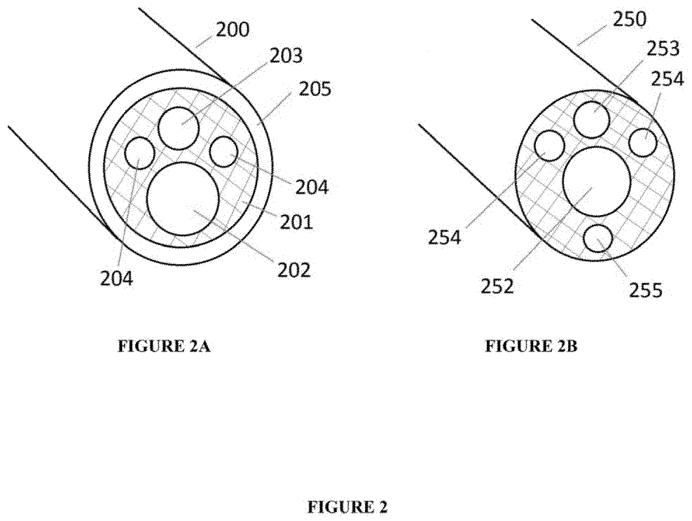

FIG. 2 shows a comparison between an embodiment 200 of an endoscope with an annular secondary channel for instillation of cleaning fluid as disclosed herein and an embodiment 250 of a circular secondary channel for instillation of cleaning fluid as described in the prior art. As shown in FIG. 2a, endoscope 200 includes an inner channel 201 that has a primary working channel 202, a lens system 203, and light sources 204, and also includes an annular secondary channel 205 consisting of a single annular fluid delivery channel for instillation of the cleaning fluid. As shown in FIG. 2b, prior art endoscope 250 has primary working channel 252, a lens system 253, light sources 254, and a circular secondary channel 255 for instillation of the cleaning fluid. As discussed above, where secondary channel 205 and secondary channel 255 have the same overall cross-sectional area, the outer diameter of endoscope 200 will be less than the outer diameter of endoscope 250. Since the maximum outer diameter of the endoscope will be constrained by its specific use, the annular design of the secondary channel allows endoscope 200 to have a wider primary working channel 202 as compared to the primary working channel 252 of endoscope 250.

Moreover, the maximum flow rate of the cleaning solution will be limited by the need to avoid tissue damage caused by the instillation of cleaning solution. If the flow rate of cleaning solution is too high, the pressure created by instillation of cleaning solution will damage the tissue being examined during endoscopy. Thus, the flow rate must be sufficient to wash away contaminants such as blood from the examination field as described above, and also must be below the level that would cause damage to the tissue being examined.

Endoscopes with examination field cleaning systems described in the prior art typically suffer from the limitation that at low fluid flow rates the surface tension of the fluid causes the fluid to exit the endoscope as droplets rather than as a spray, thus often leading to further obscuring of the examination field rather than the desired cleaning effect. To avoid droplet formation, the flow rate at which cleaning solution is instilled must exceed a threshold flow rate (Qₜ). The threshold flow rate will vary depending on the diameter of the secondary channel through which cleaning solution is instilled and also upon the design of the secondary channel. The secondary channel may be configured as an annular secondary channel 205 or as a circular secondary channel 255, as shown in FIG. 2. The annular secondary channel may include one or more fluid delivery channels, where the one or more fluid delivery channels are arranged along the circumferential edge of the endoscope to collectively form a substantially annular configuration.

The results shown in Tables 1-4 below assume that the cleaning solution is water. A saline solution or other cleaning solution will likely have highly similar properties to water for the purpose of flow rate and impact pressure calculations. Any differences between actual cleaning solutions and water with respect to the results shown in Tables 1-4 are negligible.

Table 1 shows the threshold flow rate for a circular secondary channel of specified diameter (d) and the threshold flow rate for an annular secondary channel with a single fluid delivery channel with the same cross-sectional area as the specified circular secondary channel, where four possible values for the inner diameter (dᵢ) of the annular secondary channel are specified. The outer diameter (dₒ) of the annular secondary channel that has the same cross-sectional area as the specified circular channel will vary with the diameter of the circular secondary channel, and thus the width of the annular secondary channel will vary accordingly.

TABLE 1

| | | Annular | | Annular | | Annular | | Annular | |
|---|---|---|---|---|---|---|---|---|---|
| Circular | | (dᵢ = 3 mm) | | (dᵢ = 4 mm) | | (dᵢ = 5 mm) | | (dᵢ = 6 mm) | |
| d | Qᵢ | dₒ | Qᵢ | dₒ | Qᵢ | dₒ | Qᵢ | dₒ | Qᵢ |
| (mm) | (mL/min) | (mm) | (mL/min) | (mm) | (mL/min) | (mm) | (mL/min) | (mm) | (mL/min) |
| 0.5 | 9 | 3.04 | 31 | 4.03 | 36 | 5.02 | 40 | 6.02 | 44 |
| 0.7 | 15 | 3.08 | 44 | 4.06 | 51 | 5.05 | 57 | 6.04 | 62 |
| 0.9 | 22 | 3.13 | 57 | 4.10 | 65 | 5.08 | 73 | 6.07 | 80 |
| 1.1 | 29 | 3.20 | 70 | 4.15 | 80 | 5.12 | 89 | 6.10 | 97 |
| 1.3 | 38 | 3.27 | 83 | 4.21 | 95 | 5.17 | 106 | 6.14 | 115 |
| 1.5 | 47 | 3.35 | 96 | 4.27 | 110 | 5.22 | 122 | 6.18 | 133 |
| 1.7 | 56 | 3.45 | 110 | 4.35 | 125 | 5.28 | 139 | 6.24 | 152 |
| 1.9 | 67 | 3.55 | 124 | 4.43 | 141 | 5.35 | 156 | 6.29 | 170 |
| 2.1 | 78 | 3.66 | 138 | 4.52 | 156 | 5.42 | 173 | 6.36 | 188 |
| 2.3 | 89 | 3.78 | 153 | 4.61 | 172 | 5.50 | 190 | 6.43 | 207 |
| 2.5 | 101 | 3.91 | 167 | 4.72 | 188 | 5.59 | 207 | 6.50 | 225 |
| 2.7 | 113 | 4.04 | 182 | 4.83 | 204 | 5.68 | 225 | 6.58 | 244 |
| 2.9 | 126 | 4.17 | 198 | 4.94 | 221 | 5.78 | 243 | 6.66 | 263 |
| 3.1 | 139 | 4.31 | 214 | 5.06 | 238 | 5.88 | 261 | 6.75 | 282 |

If the flow rate of the cleaning solution is below the threshold flow rate, the cleaning solution will bead up and exit the end of the secondary channel in droplets. If the flow rate of the cleaning solution is above the threshold flow rate, the cleaning solution will exit the end of the secondary channel as a spray.

As shown in Table 1, the threshold flow rate to avoid droplet formation increases with the diameter of the secondary channel. In addition, the threshold flow rate for an annular secondary channel of equivalent cross-sectional area as a circular secondary channel is always significantly higher. This indicates that preventing droplet formation will require a higher flow rate of cleaning solution when employing an annular secondary channel with a single fluid delivery channel as compared to a circular secondary channel of equal cross-sectional area.

However, as shown in Tables 2-4, the impact pressure (P) of the cleaning solution on the tissue in the examination field is significantly lower when the cleaning solution is delivered by an annular secondary channel with a single fluid delivery channel as compared to a circular secondary channel. Table 2 shows the impact pressure caused by cleaning solution delivered at a flow rate of 230 mL/min via a circular secondary channel of a specified diameter (d).

TABLE 2

| d (mm) | P (Pa) |
|---|---|
| 0.6 | 91813 |
| 0.8 | 29050 |
| 1.0 | 11899 |
| 1.2 | 5738 |
| 1.4 | 3097 |
| 1.6 | 1816 |
| 1.8 | 1133 |
| 2.0 | 744 |
| 2.2 | 508 |
| 2.4 | 359 |
| 2.6 | 260 |
| 2.8 | 194 |
| 3.0 | 147 |
| 3.2 | 113 |

Table 3 shows the flow rate (Q) for fluid instilled via an annular secondary channel with a single fluid delivery channel with a specified inner diameter (dᵢ) and outer diameter (dₒ) that is required to generate the same impact pressure as a circular secondary channel of diameter 1.0 mm instilling fluid at a flow rate of 230 mL/min, namely 11899 Pa as shown in Table 2.

TABLE 3

| | |
|---|---|
| | Annular |
| Circular | (dᵢ = 3.0 mm) |

| d (mm) | Q (mL/min) | dₒ (mm) | Q (mL/min) |
|---|---|---|---|
| 1.0 | 230 | 3.0 | 0 |
| | | 3.1 | 140 |
| | | 3.2 | 285 |
| | | 3.3 | 435 |
| | | 3.4 | 589 |
| | | 3.5 | 748 |
| | | 3.6 | 911 |
| | | 3.7 | 1079 |
| | | 3.8 | 1251 |
| | | 3.9 | 1428 |

Table 4 shows the required flow rate (Q) for fluid instilled via an annular secondary channel with a single fluid delivery channel with a specified inner diameter (dᵢ) and outer diameter (dₒ) to generate the same impact pressure as a circular secondary channel of diameter 2.0 mm instilling fluid at a flow rate of 230 mL/min, which is 744 Pa as shown in Table 2.

TABLE 4

| | | Annular | | Annular | |
|---|---|---|---|---|---|
| Circular | | (dᵢ = 3.0 mm) | | (dᵢ = 4.0 mm) | |
| d (mm) | Q (mL/min) | dₒ (mm) | Q (mL/min) | dₒ (mm) | Q (mL/mm) |
| 2.0 | 230 | 3.0 | 0 | 4.0 | 0 |
| | | 3.1 | 35 | 4.1 | 47 |
| | | 3.2 | 71 | 4.2 | 94 |
| | | 3.3 | 109 | 4.3 | 143 |
| | | 3.4 | 147 | 4.4 | 193 |
| | | 3.5 | 187 | 4.5 | 244 |
| | | 3.6 | 228 | 4.6 | 297 |
| | | 3.7 | 270 | 4.7 | 350 |
| | | 3.8 | 313 | 4.8 | 405 |
| | | 3.9 | 357 | 4.9 | 461 |
| | | 4.0 | 403 | 5.0 | 517 |
| | | 4.1 | 449 | 5.1 | 576 |
| | | 4.2 | 497 | 5.2 | 635 |

As shown in Tables 3-4, for a given dᵢ and dₒ of an annular secondary channel, generating a higher impact pressure will require a higher flow rate. The relationship is nonlinear.

As shown in Tables 3-4, the inner and outer diameter of the annular secondary channel may be adjusted to generate the same impact pressure as a circular secondary channel at a correspondingly higher flow rate. A properly designed annular secondary channel thereby allows instillation of cleaning solution at a higher rate than a circular secondary channel that would generate the same impact pressure. For any given impact pressure the annular design of the secondary channel allows a higher flow rate of cleaning solution, and thus more solution is available to clear the examination field of impurities. A high impact pressure will damage tissue in the examination field. Thus the critical threshold for instillation of cleaning solution will be not to exceed a designated critical impact pressure. By allowing more solution to enter the examination field at or below the critical impact pressure, the annular secondary channel promotes better cleaning of the examination field by instillation of cleaning solution.

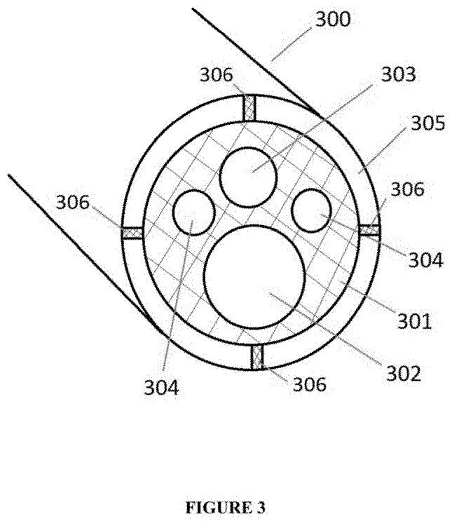

In some preferred embodiments, the endoscope may further include an anchoring bridge that secures the outer cylinder that encompasses the outer secondary channel to the tube encompassing the inner channel. FIG. 3 shows an embodiment 300 of the disclosed endoscope with an anchoring bridge, with an inner channel 301 having a primary working channel 302, a lens system 303, light sources 304, and with an outer secondary channel 305 having anchoring segments 306 which collectively form an anchoring bridge. The anchoring bridge may have three or more anchoring segments, and may preferably have four anchoring segments. The anchoring bridge may separate the outer secondary channel 305 into multiple fluid delivery channels. Water or another cleaning fluid such as an aqueous saline solution is instilled via outer secondary channel 305. The outer secondary channel 305 may be attached to a cleaning fluid storage vessel, where a valve controls the rate at which cleaning fluid enters the outer secondary channel from the cleaning fluid storage vessel. The flow rate of the cleaning fluid may optionally be controlled by a foot pedal, where the foot pedal is attached to the cleaning fluid storage vessel and where activation of the foot pedal actuates the valve. The cleaning storage vessel, valve, and foot pedal may be similar to analogous components known in the art.

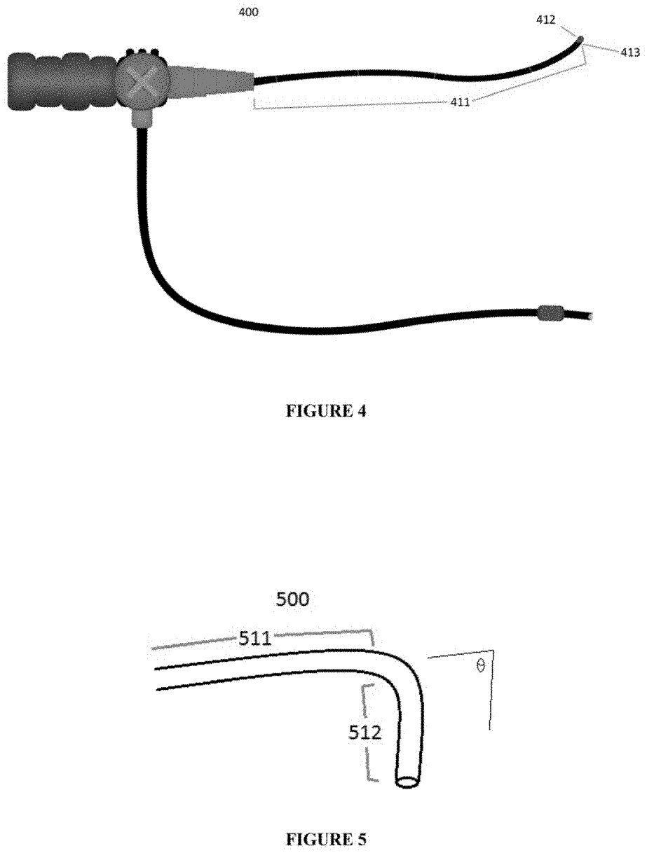

In some preferred embodiments, the endoscope may include a main body segment and a distal end segment that may be adjusted to assume an orientation relative to the main body segment, where both the inner channel and the outer secondary channel extend the entire length of the distal end segment and where the inner channel extends the entire length of the main body segment and the outer secondary channel extends at least a substantial portion of the length of the main body segment. FIG. 4 shows an embodiment 400 of the disclosed endoscope with main body segment 411 and distal end segment 412. The distal end segment may preferably extend from the distal end of the endoscope to a position about 2-3 centimeters from the distal end of the endoscope. The endoscope may further include a position adjuster 413, where the position adjuster is located at the junction between the distal end segment and the main body segment, as shown in FIG. 4. The position adjuster may allow the user to adjust the direction of the distal end segment of the endoscope, as shown in FIG. 5. The direction of the distal end segment may be aligned with the radial axis of the endoscope or may be oriented to form an angle θ between the main body segment 511 and the distal end segment 512. The angle θ may be varied using the position adjuster. By varying the angle θ, the user may insert the distal end segment into a section of the examined area that is oriented in various directions with respect to the main passage or organ through which the endoscope is inserted.

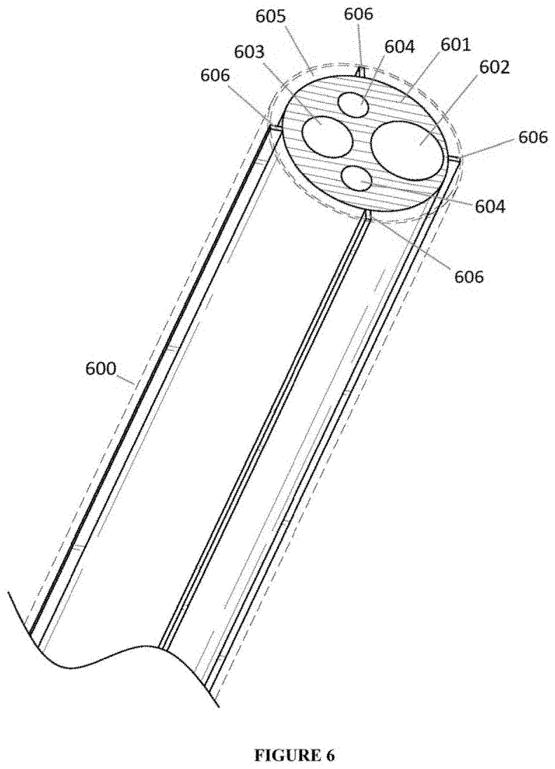

In some embodiments, the anchoring bridge may extend the entire length of the main body segment and the distal end segment. FIG. 6 shows an embodiment 600 of the disclosed endoscope with an anchoring bridge that extends the entire length of the main body segment, with an inner channel 601 having a primary working channel 602, a lens system 603, a light source 604, and with an outer secondary channel 605 having anchoring segments 606 which collectively form an anchoring bridge.

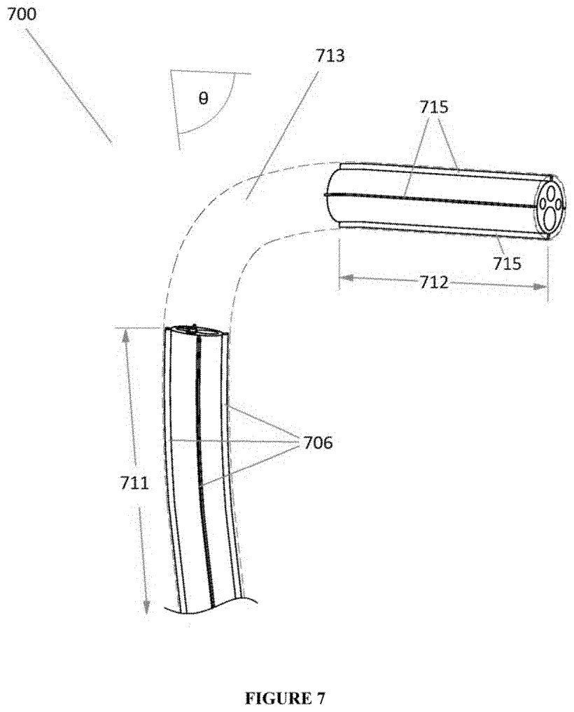

In other embodiments, the anchoring bridge may include a primary anchoring bridge segment that extends the length of the main body segment and a secondary anchoring bridge segment that extends the length of the distal end segment or that extends through some part of the distal end segment from the distal end of the endoscope. FIG. 7 shows an embodiment 700 of the disclosed endoscope having main body segment 711, distal end segment 712, and position adjustment segment 713 where primary anchoring segments 706 extend the length of the main body segment to form a primary anchoring bridge segment that extends the length of the main body segment, secondary anchoring segments 715 extend the length of the distal end segment to form a secondary anchoring bridge segment that extends the length of the distal end segment, and the position adjustment segment separates the main body segment from the distal end segment. The secondary anchoring segments may alternatively extend from the distal end of the endoscope through some part of the distal end segment but not its entire length. The distal end segment may preferably extend from the distal end of the endoscope to a position about 2-3 centimeters from the distal end of the endoscope, and the secondary anchoring segments may preferably extend between about 1 millimeter and 2-3 centimeters from the distal end of the endoscope through the distal end segment. The embodiment illustrated in FIG. 7 may allow greater flexibility and ease of adjustment of the orientation of the distal end segment with respect to the main body segment.

As shown in FIG. 3, the anchoring bridge may have anchor segments that are sufficiently narrow so as not to appreciably alter the flow characteristics of the cleaning solution through the outer secondary channel as compared to embodiments of the disclosed endoscope that do not have an anchoring bridge, such as the embodiment shown in FIG. 1. Thus the results shown in Tables 1-4 are also relevant to embodiments of the endoscope that include an anchoring bridge.

Alternatively, the anchoring bridge may have anchor segments which modify the flow characteristics of the cleaning solution through the outer secondary channel in a way that reduces the threshold flow rate (Qₜ) or that reduces the impact pressure (P) generated by a given flow rate (Q) of cleaning solution. The secondary working channel may preferably be flushed with cleaning solution before use to remove any air bubbles that would alter the flow characteristics of the cleaning solution when the endoscope is in use. This flush procedure will effectively remove any air bubbles from the secondary working channel, regardless of whether the endoscope has an anchoring bridge and regardless of whether the anchoring bridge is continuous through the length of the endoscope or whether the anchoring bridge includes a primary anchoring bridge segment and a secondary anchoring bridge segment as described above.

The anchoring bridge does not compromise the flexibility of the endoscope, and thus the outer secondary channel also has a flexibility that is similar to the flexibility of the inner channel.

Although the flow rate required to instill cleaning solution as a spray rather than as droplets when employing an annular secondary channel is higher than the corresponding flow rate for a circular channel, the width of the annular secondary channel may be adjusted to exceed the threshold flow rate without exceeding the critical impact pressure. Since the critical impact pressure may differ for different types of endoscopes, the inner diameter and width of the annular secondary channel may be tuned via routine experimentation to identify the optimal inner diameter and width of the annular secondary channel for the desired type of endoscopy. Moreover, in an endoscope having an anchoring bridge, the size and configuration of the anchoring bridge may be adjusted by modifying the size and configuration of the anchor segments, thereby modifying the flow characteristics of the cleaning solution through the outer secondary channel in a way that reduces the threshold flow rate (Qₜ) or that reduces the impact pressure (P) generated by a given flow rate (Q) of cleaning solution. Thus configuration of the annular secondary channel may be further adjusted to generate the desired flow properties of cleaning solution instilled.

Thus an annular secondary channel properly tuned to the desired type of endoscopy may obviate the limitation of droplet formation when using a circular secondary channel to instill cleaning solution.

A method of cleaning an examination field in an internal body cavity is also described herein. The internal body cavity may preferably be a human body cavity. The examination field may be cleaned using an endoscope including an inner channel that has a primary working channel, lens system, and light delivery system and an outer secondary channel that has an examination field cleaning system, where the inner channel has an approximately cylindrical shape and is encompassed by a flexible tube and the outer secondary channel is configured to be encompassed by an outer cylinder that is approximately concentric with and fully encompasses the inner channel and tube to form an annular secondary channel and where the examination field cleaning system may include one or more fluid delivery channels. The method may be employed during an endoscopy procedure to clean the examination field while it is being observed by a user using the lens system of the endoscope. A cleaning fluid may be introduced via the one or more fluid delivery channels into the examination field to wash away blood or other fluids that are obscuring the examination field. The flow rate of the cleaning fluid may optionally be controlled by a foot pedal, where the foot pedal is attached to a cleaning fluid storage vessel attached to the outer secondary channel and where a valve controls the rate at which cleaning fluid enters the outer secondary channel from the cleaning fluid storage vessel and where activation of the foot pedal actuates the valve.

The previous description of the disclosed embodiments is provided to enable any person skilled in the art to make or use the invention disclosed herein. Although the various inventive aspects are disclosed in the context of certain illustrated embodiments, implementations, and examples, it should be understood by those skilled in the art that the invention extends beyond the specifically disclosed embodiments to other alternative embodiments and/or uses of the invention and obvious modifications and equivalents thereof. In addition, while a number of variations of various inventive aspects have been shown and described in detail, other modifications that are within their scope will be readily apparent to those skilled in the art based upon reviewing this disclosure. It should be also understood that the scope of this disclosure includes the various combinations or sub-combinations of the specific features and aspects of the embodiments disclosed herein, such that the various features, modes of implementation, and aspects of the disclosed subject matter may be combined with or substituted for one another. The generic principles defined herein may be applied to other embodiments without departing from the spirit or scope of the disclosure. Thus, the present disclosure is not intended to be limited to the embodiments shown herein but is to be accorded the widest scope consistent with the principles and novel features disclosed herein.

Similarly, the disclosure is not to be interpreted as reflecting an intent that any claim set forth below requires more features than are expressly recited in that claim. Rather, as the following claims reflect, inventive aspects may reside in a combination of fewer than all features of any single foregoing disclosed embodiment.

Each of the foregoing and various aspects, together with those set forth in the claims and summarized above or otherwise disclosed herein, including the figures, may be combined without limitation to form claims for a device, apparatus, system, method of manufacture, and/or method of use.

All references cited herein are hereby expressly incorporated by reference.

What is claimed is:

1. An endoscopy apparatus comprising an endoscope, wherein the endoscope comprises:
a. an inner channel comprising:
i. a primary working channel;
ii. a lens system; and
iii. a light delivery system;
wherein the inner channel has an approximately cylindrical shape and is encompassed by a flexible inner tube;
wherein the primary working channel is configured to allow insertion of a medical instrument through the primary working channel; and
b. an outer secondary channel comprising an examination field cleaning system;
wherein the outer secondary channel is encompassed by an outer cylinder that is approximately concentric with and fully encompasses the inner tube to form an annulus around the inner tube,
wherein the outer secondary channel does not compromise the flexibility of the endoscope and the endoscope is a flexible endoscope,
wherein the examination field cleaning system comprises one or more fluid delivery channels, and
wherein the endoscope is configured for both diagnostic and therapeutic use.

2. The endoscopy apparatus of claim 1, wherein the outer secondary channel is connected to a cleaning fluid storage vessel.

3. The endoscopy apparatus of claim 2, wherein the cleaning fluid storage vessel contains a cleaning fluid.

4. The endoscopy apparatus of claim 3, wherein the cleaning fluid storage vessel is separated from the outer secondary channel by a valve that controls the rate at which the cleaning fluid enters the outer secondary channel from the cleaning fluid storage vessel.

5. The endoscopy apparatus of claim 4, wherein the cleaning fluid storage vessel is connected to a foot pedal and wherein activation of the foot pedal actuates the valve.

6. The endoscopy apparatus of claim 1, wherein the endoscope comprises a main body segment and a distal end segment, wherein the distal end segment may be adjusted to assume an orientation relative to the main body segment.

7. The endoscopy apparatus of claim 6, wherein both the inner channel and the outer secondary channel extend the entire length of the distal end segment and wherein the inner channel extends the entire length of the main body segment and the outer secondary channel extends at least a substantial portion of the length of the main body segment.

8. The endoscopy apparatus of claim 1, wherein the endoscope further comprises an anchoring bridge and wherein the anchoring bridge secures the outer cylinder to the tube encompassing the inner channel.

9. The endoscopy apparatus of claim 8, wherein the anchoring bridge comprises three or more anchoring segments.

10. The endoscopy apparatus of claim 9, wherein the anchoring bridge comprises four anchoring segments.

11. The endoscopy apparatus of claim 9, wherein the anchoring bridge separates the outer secondary channel into multiple fluid delivery channels.

12. The endoscopy apparatus of claim 6, wherein the endoscope further comprises an anchoring bridge and wherein the anchoring bridge secures the outer cylinder to the tube encompassing the inner channel.

13. The endoscopy apparatus of claim 12, wherein the anchoring bridge extends the entire length of the main body segment and the distal end segment.

14. The endoscopy apparatus of claim 8, wherein the anchoring bridge comprises a primary anchoring bridge segment that extends the length of the main body segment and a secondary anchoring bridge segment that extends the length of the distal end segment or that extends through some part of the distal end segment from the distal end of the endoscope.

15. A method of cleaning an examination field in an internal body cavity using the endoscopy apparatus of claim 1.

16. The method of claim 15, wherein the outer secondary channel of the endoscope is connected to a cleaning fluid storage vessel.

17. The endoscopy apparatus of claim 16, wherein the cleaning fluid storage vessel contains a cleaning fluid.

18. The endoscopy apparatus of claim 17, wherein the cleaning fluid storage vessel is separated from the outer secondary channel by a valve that controls the rate at which the cleaning fluid enters the outer secondary channel from the cleaning fluid storage vessel, wherein the cleaning fluid storage vessel is connected to a foot pedal, and wherein activation of the foot pedal actuates the valve.

19. The endoscopy apparatus of claim 1, wherein the endoscope is a bronchoscope.

20. The endoscopy apparatus of claim 14, wherein the endoscope is a bronchoscope.
